(19) United States
(12) Patent Application Publication
(10) Pub. No.: US 2009/0118776 A1
(43) Pub. Date: May 7, 2009

(54) Tissue anchors

(75) Inventors: Daniel N. Kelsch, Fairview Park, OH (US); Kenneth P. Rundle, Independence, OH (US)

Correspondence Address: PEARNE & GORDON LLP, 1801 EAST 9TH STREET, SUITE 1200, CLEVELAND, OH, 44114, US

(73) Assignee: Biomec, Inc., Cleveland, OH (US)

(21) Appl. No.: 11/232,182

(22) Filed: Sep. 21, 2005

Related U.S. Application Data
(60) Provisional application No. 60/613,004, filed on Sep. 24, 2004.

(51) Int. Cl. A61B 17/04; A61B 17/08

(52) U.S. Cl. 606/325; 606/151; 606/300

(57) ABSTRACT

Tissue anchors comprise a head and a fastening structure attached to the head. In one example, the fastening structure can include a helical structure disposed about an axis with a first end portion attached to the head and a second end portion including a distal end of the helical structure.

### CROSS-REFERENCE TO RELATED APPLICATION

The present invention claims the benefit of U.S. Provisional Application No. 60/613,004 filed Sep. 24, 2004, the entire disclosure which is herein incorporated by reference.

### FIELD OF THE INVENTION

The present invention relates to anchors and, more particularly, to tissue anchors for attaching elements to a tissue structure.

### BACKGROUND OF THE INVENTION

It is generally known to provide an anchor to fasten connective tissue to bone. Conventional anchors are commonly provided as a screw with a helical thread or an open helical structure. Conventional screw-type anchors typically require predrilling of relatively large holes in the bone and can therefore substantially weaken the overall bone structure. Moreover, conventional anchors with an open helical structure may not have a desired level of structural integrity for certain applications and/or may be expensive or difficult to produce.

There is a need for durable, strong anchors that are structurally sound and relatively inexpensive to produce.

### SUMMARY OF THE INVENTION

In accordance with one aspect, an anchor is provided for mounting to a tissue structure. The anchor comprises a head including a shroud encircling an interior area. The shroud includes an outer periphery with two substantially flat surfaces that are substantially parallel with respect to one another. The head further includes a crossbar having first and second ends attached to the shroud such that the crossbar extends within the interior area between the two substantially flat surfaces. The anchor further includes a fastening structure attached to the head and configured for mounting to a tissue structure.

In accordance with another aspect, a helical anchor is provided with a head and a helical structure. The helical structure is disposed about an axis and includes a first end portion attached to the head and a second end portion including a distal end of the helical structure. The helical structure includes a cross section that decreases from the first end portion to the second end portion. Perimeters of each cross section are mathematically similar to one another.

In accordance with still another aspect, a helical anchor is provided with a head and a helical structure. The helical structure has a length and is disposed about an axis. The helical structure includes a first end portion attached to the head and a second end portion including a distal end of the helical structure. The helical structure further includes a cross section and an axial thickness that each decrease from the first end portion to the second end portion.

It is to be appreciated that other, different, possibly more broad aspects are provided as other aspects of the present invention.

### BRIEF DESCRIPTION OF THE DRAWINGS

The foregoing and other aspects of the present invention will become apparent to those skilled in the art to which the present invention relates upon reading the following description with reference to the accompanying drawings, in which:

FIG. 1 depicts a perspective view of a helical anchor in accordance with a first embodiment of the present invention;

FIGS. 2-4 are side views of the helical anchor of FIG. 1;

FIG. 5 is a bottom view of the helical anchor of FIG. 1;

FIG. 6 is a top view of the helical anchor of FIG. 1;

FIG. 7 is a sectional view of the helical anchor along line 7-7 of FIG. 6;

FIG. 8 is a perspective view of a helical anchor in accordance with a second embodiment of the present invention;

FIGS. 9 and 10 are side views of the helical anchor of FIG. 8;

FIG. 11 is a bottom view of the helical anchor of FIG. 8;

FIG. 12 is a top view of the helical anchor of FIG. 8;

FIG. 13 is a sectional view of the helical anchor along line 13-13 of FIG. 12;

FIGS. 14 and 15 are side views of a helical anchor in accordance with a third embodiment of the present invention;

FIG. 16 is a sectional view of the helical anchor along line 16-16 of FIG. 14;

FIG. 17 is a top view of the helical anchor viewed along line 17-17 of FIG. 15;

FIG. 18 is a bottom view of the helical anchor of FIG. 14;

FIG. 19 is side view of a portion of the helical anchor of FIG. 14;

FIG. 20 is a perspective view of a helical anchor in accordance with a fourth embodiment of the present invention;

FIGS. 21 and 22 are side views of the helical anchor of FIG. 20;

FIG. 23 is a bottom view of the helical anchor of FIG. 20;

FIG. 24 is a top view of the helical anchor of FIG. 20; and

FIG. 25 is a sectional view of the helical anchor along line 25-25 of FIG. 24.

### DETAILED DESCRIPTION OF EXAMPLE EMBODIMENTS

Certain terminology is used herein for convenience only and is not to be taken as a limitation on the present invention. Further, in the drawings, the same reference numerals are employed for designating the same elements.

Anchors in accordance with the present invention are adapted to fasten an element with respect to a tissue structure (e.g., an organic or artificial tissue structure). The tissue structure can comprise, for example, organic bone tissue. In further examples, the tissue structure can comprise an artificial tissue structure, such as artificial bone tissue, a prosthetic or other artificial device functioning in place of, or in addition to, organic tissue. Examples of an element can comprise another tissue structure (e.g., an artificial or organic tissue as described above). For instance, elements may comprise a muscle tissue, a connective tissue (e.g., tendons, ligaments, cartilage, etc.), a suture, a portion of a prosthetic or other artificial device, or the like. In one application, anchors can be adapted to fasten a suture to organic bone tissue. Once fastened, the suture and anchor combination can then be used to attach a muscle tissue or connective tissue to the organic bone tissue.

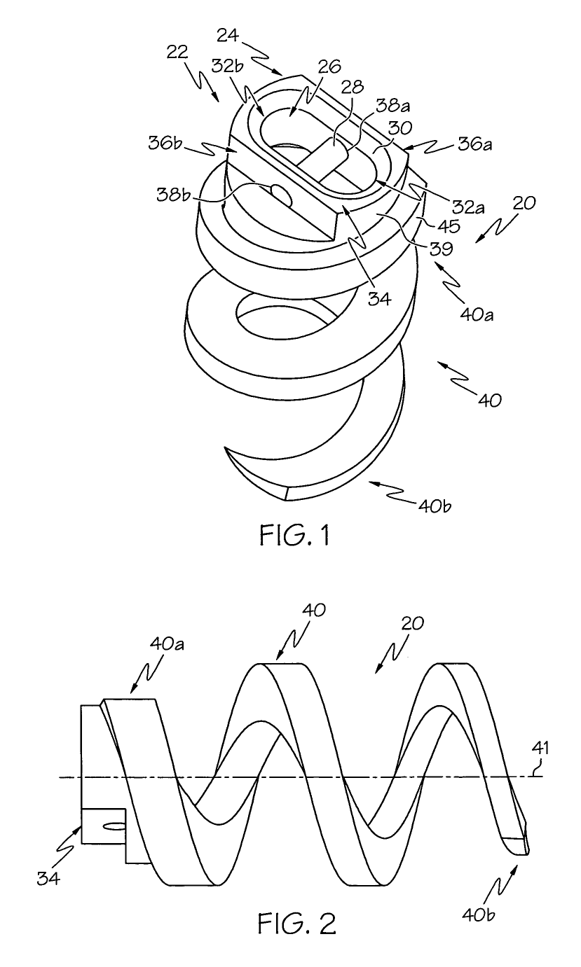

One example of a helical anchor 20 is illustrated in FIGS. 1-7. As shown in the perspective view of FIG. 1, the helical anchor 20 can include a head 22 with a shroud 24 encircling an interior area 26 adapted to protect portions of an element such as a suture. The head 22 can further include a crossbar 28 adapted to provide a fastening location for the element (e.g., suture).

The head 22 may also be designed to inhibit, such as prevent, undue wear such abrading and/or cutting of the element against surfaces of the head 22. For example, the crossbar 28 may be formed with one or more rounded surfaces to present a low friction mounting location. In the illustrated example, the crossbar 28 can comprise a cylindrical bar having a generally circular cross section to provide a continuous smooth surface for the suture or other element engaging the crossbar 28. In further embodiments, the crossbar can comprise one or more nonrounded surfaces. For instance, the crossbar may be formed with noncircular cross sections such as a substantially polygonal or other cross sectional shapes. If a polygonal cross section is provided, the corners of the polygon can be rounded to inhibit wearing of the element (e.g., suture) contacting the crossbar. In further embodiments, the cross bar may have nonrounded surfaces, for instance, where the element comprises a durable material (e.g., metal wire or cable) or where there is little or no relative movement between the element and the anchor.

The shroud 24 may also be designed to inhibit, such as prevent, undue wear such abrading and/or cutting of the element. For example, the shroud 24 can include a rounded inner periphery 30 adjacent the entrance to the interior area 26. As shown, the rounded inner periphery 30 can extend substantially continuously about the inner periphery of the interior area 26 to reduce or prevent exposure of the element to sharp corner edges. Still further, the head may include interior arcuate wall portions 32a, 32b designed to further reduce interior sharp corner edges. As shown arcuate wall portions 32a, 32b provided the interior area 26 with a substantially oblong shape.

The head 22 may also be designed to minimize or eliminate pinch points for portions of the element contacting the crossbar; thereby, further inhibiting undue wear such as abrading and/or cutting of the element. For example, as shown, the shroud 24 encircles the interior area 26 to protect portions of an element, such as a suture, that can be attached to the crossbar 28. Therefore, pinching of the element (e.g., suture) between the crossbar 28 and an adjacent structure (e.g., tissue structure) is inhibited or prevented by the protective shroud 24. In one example, a suture can be tied about the outer periphery of the crossbar 28 wherein tied portions of the suture are located within the interior area 26. Locating the tied and/or other portions of the suture within the interior area allows the shroud 24 to act as a barricade to isolate portions of the suture from adjacent structures (e.g., tissue structures). The shroud 24 may therefore inhibit, such as prevent, failure of the suture by inhibiting adjacent structures from pinching the suture between the adjacent structure and the crossbar 28. In addition or alternatively, as further illustrated in FIG. 7, the crossbar 28 can be offset from a bottom surface 23 of the shroud 24 by a distance “D”. Offsetting the crossbar 28 from the bottom surface can further protect the suture from being pinched between the tissue structure (e.g., bone tissue) and a surface of the anchor 20 as the anchor is being attached to the tissue structure.

In one example, the head 22 is designed to protect portions of the element (e.g., suture) from a sharp bone surface or edge located adjacent the anchor. For example, the anchor 20 may be sufficiently screwed into the bone tissue such that the head 22 is substantially countersunk into the bone tissue. For instance, the head 22 can be countersunk in the bone tissue such that a top surface 34 of the head 22 is substantially flush or adjacent to the outer surface of the bone tissue. In this instance, the shroud 24 protects the element (e.g., suture) from engaging the sharp edges of the bone tissue located adjacent the crossbar 28. Instead of rubbing up against the sharp edges of the bone, the element (e.g, suture) would only be exposed to the rounded inner periphery 30 of the shroud 24, thereby reducing exposure to sharp bone edges that might otherwise occur without the shroud 24. At the same time, the offset location of the crossbar 28 from the bottom surface 23 of the shroud 24 protects the suture from being pinched between the tissue structure (e.g., bone tissue) as the anchor 20 is screwed into the bone tissue.

The head 22 may further include structure adapted to engage a mounting tool to transfer torque from the tool to the anchor as the anchor is screwed into the bone tissue or other tissue structure. In one example, the head 22 can include at least two opposed substantially flat, parallel surfaces 36a, 36b adapted to provide an engagement surface for the tool. In one example, the tool can comprise two opposed prongs adapted to simultaneously engage the flat, parallel surfaces 36a, 36b. Two surfaces can be desirable to inhibit relative slip or other failed engagement between the head 22 and the tool. Although two substantially flat, parallel surfaces are shown, three or more surfaces may be provided in additional embodiments. As further shown, the substantially flat, parallel surfaces 36a, 36b may each comprise an aperture 38a, 38b adapted to receive a corresponding end of the crossbar 28 for fastening the crossbar relative to the shroud 24. In the illustrated example, the crossbar 28 is located to extend within the interior area 26 between the first and second substantially flat surface 36a, 36b. Extending the crossbar 28 between the surfaces 36a, 36b can strengthen the head from a compression force acting between the surfaces 36a, 36b. Thus, if the mounting tool comprises a clamping tool, significantly higher clamping forces may be applied between the surfaces 36a, 36b without permanently deforming the head 22 of the helical anchor 20.

As shown in FIGS. 1 and 2, the anchor 20 includes at least one helical structure 40 disposed about an axis 41 and extending from the head 22. The helical structure 40 begins at a first end portion 40a attached to the head 22 and ends at a second end portion 40b. In one example, the helical structure is only attached to the bottom surface of the anchor head. In further embodiments, as shown in FIGS. 1 and 7, the first end portion 40a of the helical structure 40 is attached at least partially to an outer periphery 39 of the head 22 rather than only to the bottom surface of the anchor head. Attaching the helical structure 40 at least partially to the outer periphery of the anchor head can increase the strength of the connection between the helical structure 40 and the head 22.

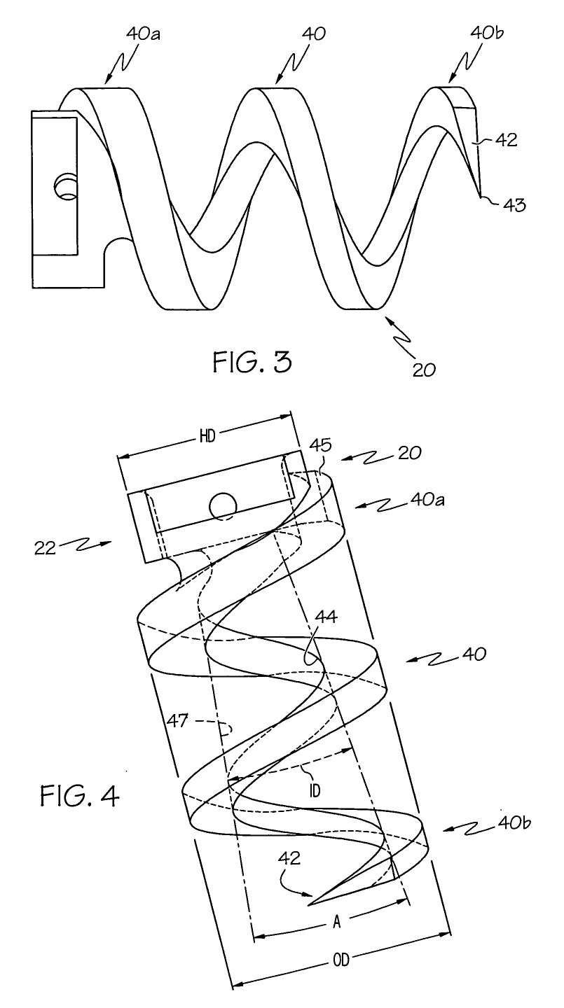

The helical structure can also extend outwardly from an outer periphery of the anchor head. For example, as shown in FIGS. 1, 4, 6 and 7, the helical structure 40 can be at least partially attached to the outer periphery 39 of the head 22 such that at least a part 45 of the first end portion 40a extends outwardly from the outer periphery 39. Such a construction can provide a helical structure with an outer diameter that is greater than a maximum dimension of the head. For instance, as shown in FIG. 4, the helical structure 40 can include an outer diameter “OD” that is greater than a head dimension “HD”, including a maximum head direction in a direction of the outer diameter “OD”. Providing the helical structure with an outer diameter that is greater than the maximum head dimension can help further isolate the element (e.g., suture) from the tissue structure (e.g., bone tissue) and can help countersink the head with respect to the outer surface of the tissue structure.

As shown in FIGS. 4 and 7, the outer diameter “OD” of the helical structure 40 can include a substantially constant outer diameter throughout a substantial portion the helical flight. The helical structure can also include an inner diameter that, in some embodiments, may comprise a substantially constant inner diameter throughout a substantial portion of the helical flight. Further embodiments include an inner diameter that changes from the throughout a substantial portion of the helical flight. For instance, as shown in FIG. 4, the helical structure 40 includes an inner diameter “ID” that substantially continuously increases in diameter in a direction from the first end portion 40a to the second end portion 40b of the helical structure 40. As shown, the continuously increasing inner diameter “ID” of the helical structure 40 forms a frustoconical helical surface 44 as shown in FIG. 4. The frustoconical helical surface 44 defines a frustoconical cavity 47 with a taper angle “A”. Various taper angles “A” may be provided in accordance with aspects of the present invention. In one example, a taper angle “A” can comprise about 10° although other taper angles may be provided in further embodiments.

In examples of the invention, the cross sectional area of the helical structure can remain substantially constant throughout a substantial portion of the helical flight. In further embodiments, the cross sectional area of the helical structure can include at least a portion that reduces in cross section along the helical flight in a direction from the first end portion to the second end portion of the helical structure. In still further embodiments, at least a portion of the flight reduces in cross section substantially continuously along the helical flight in a direction from the first end portion to the second end portion of the helical structure.

As shown in FIG. 13, substantially the entire flight of the helical structure 40 reduces in cross section substantially continuously along the helical flight in a direction from the first end portion 40a to the second end portion 40b of the helical structure 40. Providing a reduction in cross sectional area eases the anchoring process by first introducing a relatively small cross section to be received by the tissue structure (e.g., bone tissue) with a gradually increasing cross sectional area as the anchor is screwed into the tissue structure. Still further, the structural integrity of the helical structure 40 can be enhanced by providing the helical structure 40 with a relatively large cross sectional area at the mounting location between the helical structure 40 and the head 22. Moreover, providing a substantial continuous change in cross section can also reduce stress point concentrations along the helical structure and can also produce a wedging effect between the helical structure and the tissue structure. The wedging effect can facilitate screwing of the anchor structure into the tissue structure and can also help inhibit, such as prevent, subsequent loosening or disengagement of the helical structure from the tissue structure.

The helical structure can include a wide range of structural features to provide a reduced cross section along a portion of the helical flight in a direction from the first end portion to the second end portion of the helical structure. For example, the inside and outside diameters may change at different rates relative to one another in a direction from the first end portion to the second end portion of the helical structure. As shown in FIG. 4, the outer diameter “OD” can remain substantially constant throughout a portion of the helical flight of the helical structure 40 while the inner diameter “ID” of the helical structure continuously increases in diameter throughout the same portion of the helical-flight in a direction from the first end portion 40a to the second end portion 40b of the helical structure 40. In this circumstance, the radial thickness “W” of the helical structure reduces substantially continuously along a substantial portion of the helical flight from the first end portion 40a to the second end portion 40b of the helical structure 40. For instance, as illustrated in FIG. 7, radial thickness “W₁” is greater than radial thickness “W₂” which is greater than radial thickness “W₃” which is also greater than radial thickness “W₄”. Accordingly, providing a substantially constant outer diameter and an inner diameter that substantially continuously increases can result in a substantially continuous reduction in radial thickness “W” of the helical structure 40 along the helical flight from the first end portion 40a to the second end portion 40b.

As shown in FIG. 7, the substantially continuous reduction in radial thickness “W” can contribute to a substantially continuous reduction in cross sectional area “C” along a portion of the helical flight in a direction from the first end portion 40a to the second end portion 40b of the helical structure 40. For example, as shown in FIG. 7, the substantially continuous reduction in radial thickness “W” along the helical structure 40 contributes to successively reduced cross sections “C” wherein the cross section “C₁” is greater than the cross section “C₂” which is greater than cross section “C₃” which is also greater than cross section “C₄”.

In addition, or alternatively, an axial thickness “H” of the helical structure 40 may be reduced along the flight to change the cross sectional area of the flight. For example, as shown, the axial thickness “H” of the helical structure 40 can be reduced (e.g., substantially continuously reduced) along a portion of the helical flight (e.g., a substantial portion of the helical flight) in a direction from the first end portion 40a to the second end portion 40b of the helical structure 40. Indeed, as illustrated in FIG. 7, the axial thickness “H₁” is greater than the axial thickness “H₂” which is greater than the axial thickness “H₃” which is also greater than the axial thickness “H₄”. Accordingly, the cross section “C” of the helical flight can be reduced by reducing (e.g., substantially continuously reducing) the height “H” and/or the width “W” of the helical flight along a portion of the helical flight (e.g., a substantial portion of the helical flight).

Still further, the reduced cross sections, if provided, can have similar perimeters, in the mathematical sense. For example, as shown in FIG. 13, each cross section “C₁”, “C₂”, “C₃” and “C₄” has successively reduced cross sections that have similar perimeters. For example, the perimeters can be “similar” in the mathematical sense if each perimeter forms a substantially polygonal shape having three or more sides that are angularly offset from one another wherein each of the corresponding three or more angles are congruent and all corresponding sides are proportional. Perimeters can also be viewed as mathematically similar if the perimeters are simply enlarge versions of one another. Therefore, it is possible to provide cross sections with perimeters that have the same shape (e.g., polygonal or nonpolygonal) but have successively reduced sizes. The particular cross sectional shape may also be selected to reduce stress concentrations. Accordingly, providing cross sections having similar perimeters may provide an optimal cross sectional perimeter shape at substantially each location along the helical flight to reduce undesirable bending and shear stress concentrations that can otherwise develop with cross sections having an undesirable perimeter shape. In the illustrated example, each cross section “C₁”, “C₂”, “C₃” and “C₄” is formed by mathematically similar polygonal shapes having four sides. Although embodiments of the present invention illustrate polygonal structures having four sides, it is contemplated that the embodiments herein can include polygonal structures with three or more sides and might include other nonpolygonal perimeter shapes.

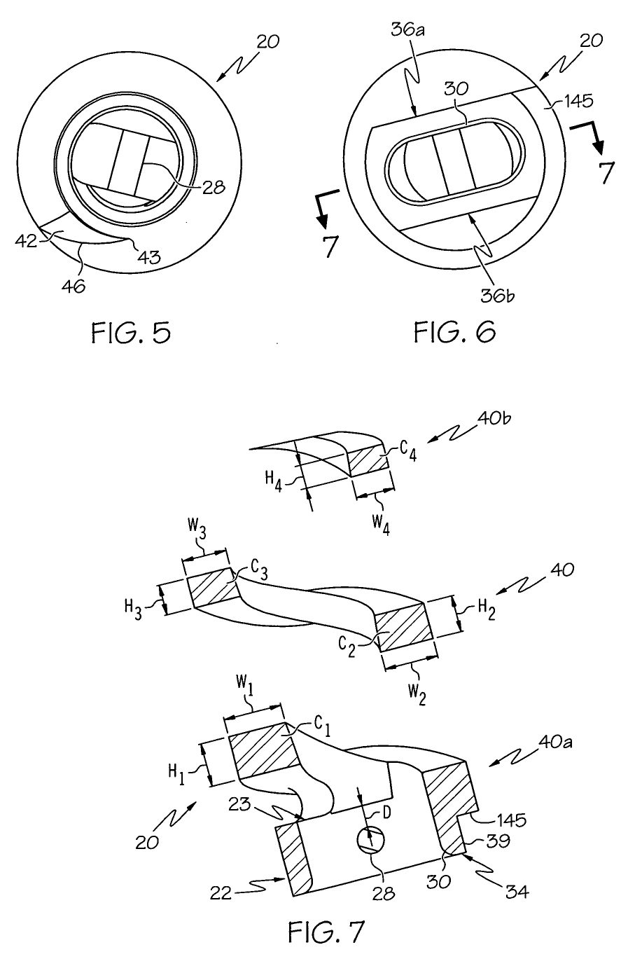

The second end portion 40b of the helical structure 40 can also include a distal end 42 to facilitate penetration and/or feeding of the helical structure 40 within the tissue structure. For example, as shown in FIG. 3, the distal end 42 can be provided with a tip 43 to optimize initial penetration and subsequent feeding of the helical structure 40 into the tissue structure. As further illustrated, the distal end 42 can be provided with an initial pitch, for example a 3° pitch, to present the tip 43 at the outermost location of the anchor 20. As shown in FIG. 5, the distal end 42 can also include a curved blade portion 46 to further facilitate initial penetration and subsequent feeding of the helical structure 40 into the tissue structure.

FIGS. 8-13 illustrate views of a helical anchor 120 in accordance with a second embodiment of the present invention. The helical anchor 120 can include similar and/or identical features described with respect to the helical anchor 20 illustrated in FIGS. 1-7 described above. For example, as shown in the perspective view of FIG. 8, the helical anchor 120 can include a head 122 including a shroud 124 encircling an interior area 126 adapted to protect portions of an element such as a suture. The head 122 can further include a crossbar 128 adapted to provide a fastening location for the element (e.g., suture). The crossbar 128 can comprise one or more structures that are similar to and/or identical to the crossbar 28 described above. For example, as shown, the crossbar 128 can comprise a cylindrical bar having a generally circular cross section to provide a continuous smooth surface for the suture or other element engaging the crossbar 128. While the inner periphery 30 of the anchor head 20 depicted in FIGS. 1-7 comprise a general oval shape, it is possible to provide the inner periphery with a circular or other shape. In one example, the shapes may provide smooth curves or transitions to reduce snag points or other locations that may accelerate wear of the element (e.g., suture). In the embodiment illustrated in FIGS. 8-13, for example, the inner periphery 130 comprises a general circular shape. Although not shown, the inner periphery 130 may also comprise a rounded inner periphery, similar to the rounded inner periphery 30, to reduce or prevent exposure of the element to sharp corner edges.

As with the shroud 24, the shroud 124 of the anchor 120 can inhibit, such as prevent, undue wear such as abrading and/or cutting of the element from foreign objects that can otherwise contact the crossbar 128. In addition, by providing a rounded inner periphery 130, the head 122 may also inhibit, such as prevent, undue wear of the element by a sharp bone surface or edge located adjacent the anchor. Indeed, as with the anchor 20, the anchor 120 may be sufficiently screwed into the tissue structure such that the head 122 is substantially countersunk into the tissue structure. As described above, providing a rounded inner periphery 130 can protect the element from engaging adjacent sharp edges of bone tissue.

Still further, as with the head 22, the head 122 can include two opposed substantially flat surfaces 136a, 136b adapted to provide an engagement surface for a tool designed to provide torque to the anchor 120 to mount the anchor 120 to the bone tissue or other tissue structure. Although two substantially flat, parallel surfaces are shown, three or more surfaces may be provided in additional embodiments. As further shown, the substantially flat, parallel surfaces 136a, 136b may each comprise an aperture 138a, 138b adapted to receive a corresponding end of the crossbar 128 for fastening the crossbar relative to the shroud 124. In the illustrated example, the crossbar 128 is located to extend within the interior area 126 between the first and second substantially flat surface 136a, 136b. Extending the crossbar 28 between the surfaces 136a, 316b can strengthen the head from a compression force acting between the surfaces 136a, 136b.

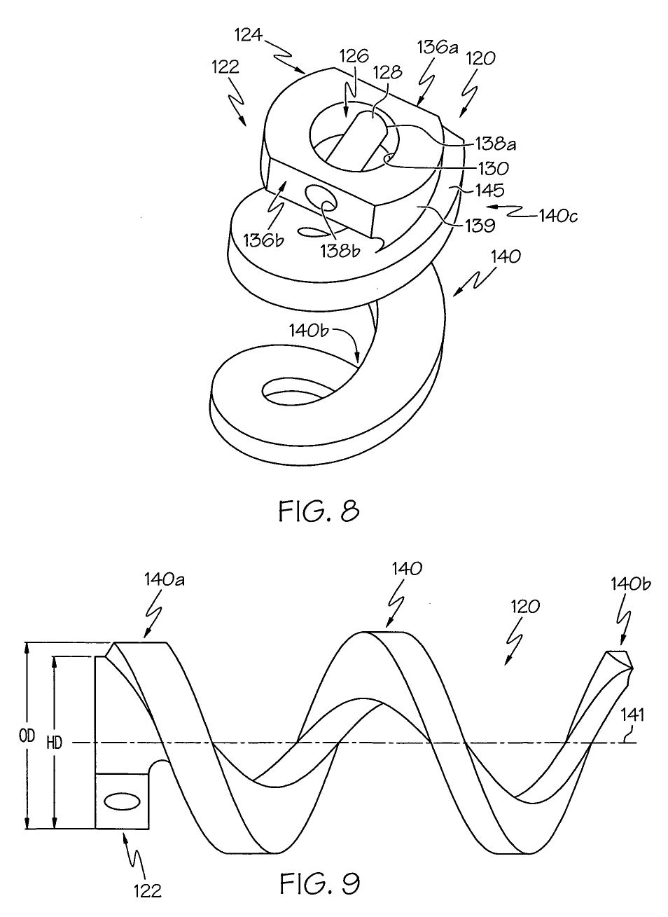

As shown in FIGS. 8 and 9, the anchor 120 includes at least one helical structure 140 disposed about an axis 141 and extending from the head 122. The helical structure 140 begins at a first end portion 140a attached to the head 122 and ends at a second end portion 140b. In one example, the helical structure is only attached to the bottom surface of the anchor head. In further embodiments, as shown in FIGS. 8 and 9, the first end portion 140a of the helical structure 140 is attached at least partially to an outer periphery 139 of the head 122 rather than only to the bottom surface of the anchor head. Attaching the helical structure 140 at least partially to the outer periphery 139 of the anchor head 122 can increase the strength of the connection between the helical structure 140 and the head 122.

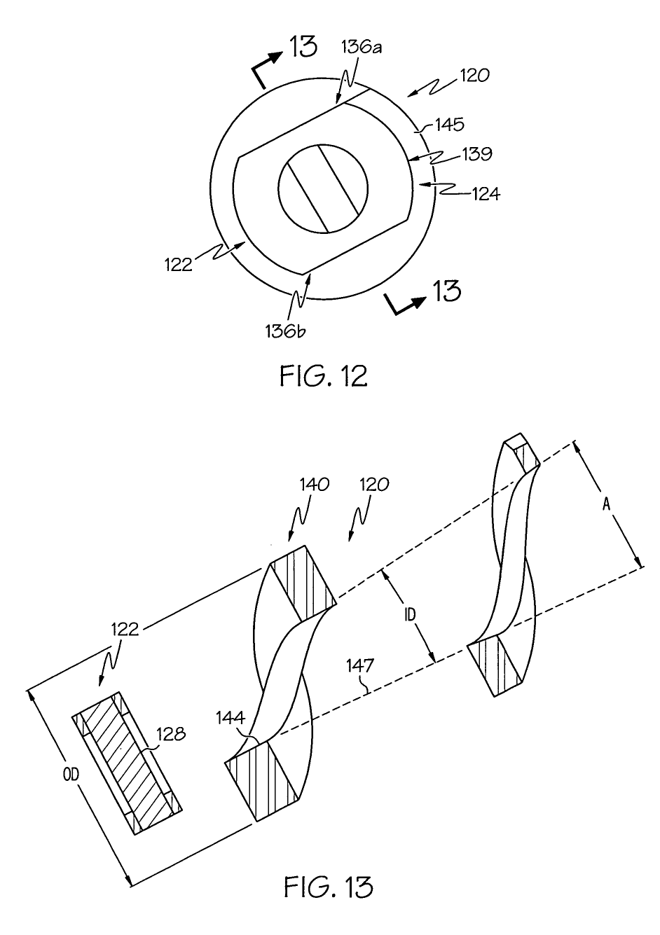

As with the helical structure 40 described above, the helical structure 140 can extend outwardly from an outer periphery 139 of the anchor head 122. For example, as shown in FIGS. 8 and 12, the helical structure 140 can be at least partially attached to the outer periphery 139 of the head 122 such that at least a part 145 of the first end portion 140a extends outwardly from the outer periphery 139. Such a construction can provide a helical structure 140 with an outer diameter that is greater than a dimension of the head 122 as described with respect to the helical structure 40.

As shown in FIGS. 8 and 9, the outer diameter “OD” of the helical structure 140 can include a substantially constant outer diameter throughout a substantial portion the helical flight. The helical structure can also include an inner diameter that, in some embodiments, may comprise a substantially constant inner diameter throughout a substantial portion of the helical flight. Further embodiments include an inner diameter that substantially continuously changes throughout a substantial portion of the helical flight. For instance, as shown in FIG. 13, the helical structure 140 includes an inner diameter “ID” that substantially continuously increases in diameter in a direction from the first end portion 140a to the second end portion 140b of the helical structure 140. As shown in FIG. 13, the continuously increasing inner diameter “ID” of the helical structure 140 forms a frustoconical helical surface 144 that defines a frustoconical cavity 147 with a taper angle “A”. Various taper angles “A” may be provided in accordance with aspects of the present invention. The taper angle “A” of the frustoconical cavity 147 can comprise about 10° although other taper angles may be provided in further embodiments.

As with the helical structure 40, substantially the entire flight of the helical structure 140 can reduce in cross section substantially continuously along a substantial portion of the flight in a direction from the first end portion 140a to the second end portion 140b. As shown, the outer diameter “OD” can remain substantially constant throughout a portion of the flight of the helical structure 140 while the inner diameter “ID” of the helical structure 140 continuously increases in diameter throughout the same portion of the flight in a direction from the first end portion 140a to the second end portion 140b. As with the radial thickness “W” of the helical structure 40, the radial thickness of the helical structure 140 can reduce substantially continuously along a substantial portion of the helical flight from the first end portion 140a to the second end portion 140b. Likewise, the substantially continuous reduction in radial thickness of the helical structure 140 can contribute to a substantially continuous reduction in cross sectional area along a portion of the helical flight in a direction from the first end portion 140a to the second end portion 140b.

In addition, or alternatively, other dimensions may affect the cross sectional area of the flight. For example, as with the axial thickness “H” of the helical structure 40, an axial thickness of the helical structure 140 may be reduced along the flight to change the cross sectional area of the flight. For example, as shown, the axial thickness of the helical structure 140 can be reduced (e.g., substantially continuously reduced) along a portion of the helical flight (e.g., a substantial portion of the helical flight) in a direction from the first end portion 140a to the second end portion 140b of the helical structure 140. Accordingly, the cross section of the helical flight 140 can be reduced by reducing (e.g., substantially continuously reducing) the height “H” and/or the width “W” of the helical flight along a portion of the helical flight.

Still further, as with the helical flight 40, the reduced cross sections of the helical flight 140, if provided, can have similar perimeters, in the mathematical sense. For example, as shown in FIG. 13, each cross section has successively reduced cross sections that have similar perimeters. In the illustrated example, each cross section of the helical structure 140 is formed by mathematically similar polygonal shapes having four sides. Although embodiments of the present invention illustrate polygonal structures having four sides, it is contemplated that the embodiments herein can include polygonal structures with three or more sides and might include other nonpolygonal perimeter shapes.

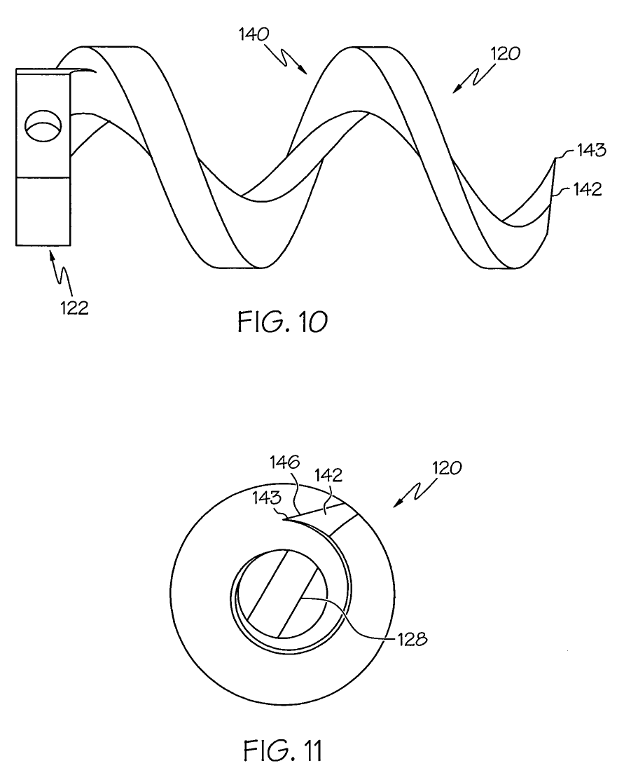

The second end portion 140b of the helical structure 140 can also include a distal end 142 to facilitate penetration and/or feeding of the helical structure 140 within the tissue structure. For example, as shown in FIG. 10, the distal end 142 can be provided with a tip 143 to optimize initial penetration and subsequent feeding of the helical structure 140 into the tissue structure. As further illustrated, the distal end 142 can be provided with an initial pitch, for example a 3° pitch, to present the tip 143 at the outermost location of the anchor 120. As shown in FIG. 11, the distal end 42 can further include a curved blade portion 146 to further facilitate initial penetration and subsequent feeding of the helical structure 140 into the tissue structure.

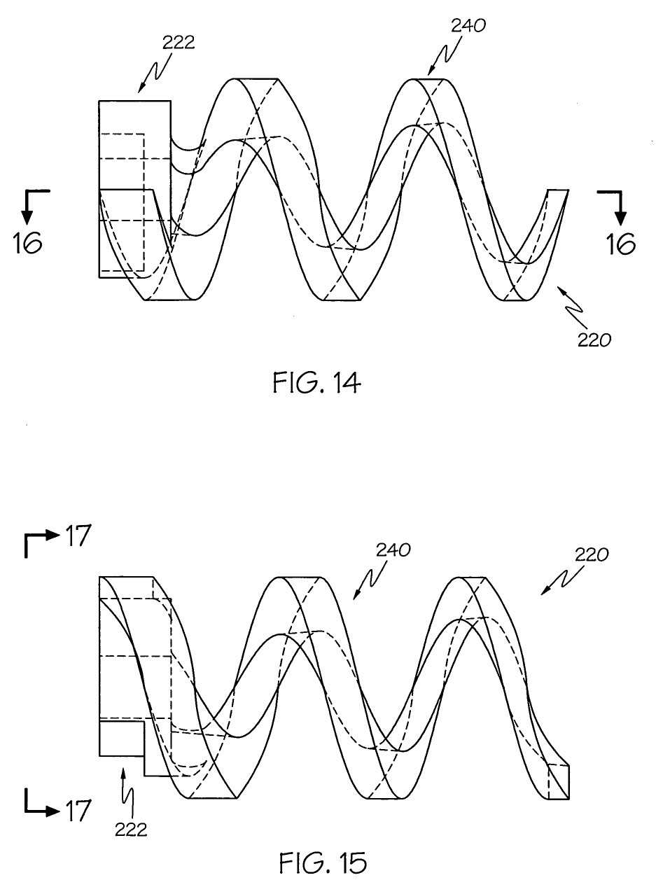

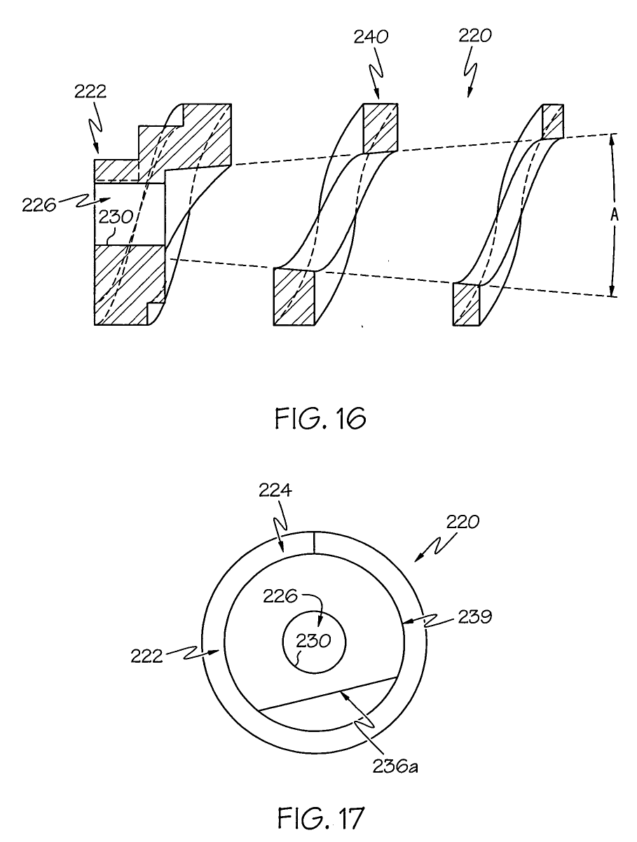

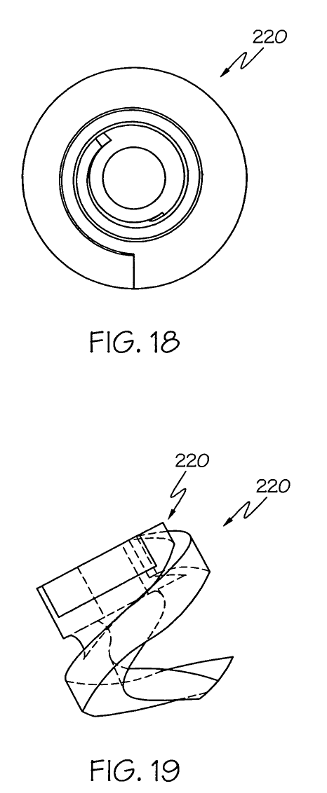

FIGS. 14-19 illustrate views of a helical anchor 220 in accordance with a third embodiment of the present invention. The helical anchor 220 can include similar and/or identical features described and illustrated with respect to the helical anchor 20 and/or the helical anchor 120. For example, the helical anchor 220 can include a helical structure 240 that can be similar or identical to helical structures described and illustrated with respect to the helical structures 40 and/or 140. The helical anchor 220 can be used as shown or may be provided as a blank for forming other, more refined anchors. For example, an initial process may be used to produce the helical anchor 220 wherein subsequent machining techniques may be used to produce the anchor 20 (i.e., see FIGS. 1-7) from the rough anchor 220 shown in FIGS. 14-19.

The helical anchor 220 illustrates a head 222 including similar and/or identical features described and illustrated with respect to the head 122 of the helical anchor 120. As shown in FIG. 17, the head 222 can include a single substantially flat surface 236a although two or more substantially flat surfaces may be provided in further embodiments. For example, a subsequent machining technique may be performed to provide two substantially flat, parallel surfaces 36a, 36b as shown in FIGS. 1 and 6. If the helical anchor 220 is only provided with a single substantially flat surface, a mounting tool may be designed to engage an outer periphery 239, including the single substantially flat surface 236a, to transfer torque from the tool to the helical anchor 220 as the helical anchor 220 is screwed into the bone tissue or other tissue structure. Other than the single substantially flat surface 236a, the head 222 can include a shroud 224 with similar and/or identical features described and illustrated with respect to the shroud 124 of the helical anchor 120 described above. For example, the shroud 224 can encircle an interior area 226 adapted to protect portions of an element such as a suture. As shown, the inner periphery 230 of the interior area 226 can comprise various shapes, such as the illustrated circular shape. The inner periphery 230 may also be subsequently machined into other shapes, such as the oval shape illustrated with respect to the inner periphery 30 of the helical anchor 20. Moreover, further machining techniques may be used to round the periphery 230 to achieve the rounded inner periphery 30 of the helical anchor 20 described above. Still further, additional machining techniques may be used to provide apertures in the shroud 224 to received corresponding ends of a crossbar as described above.

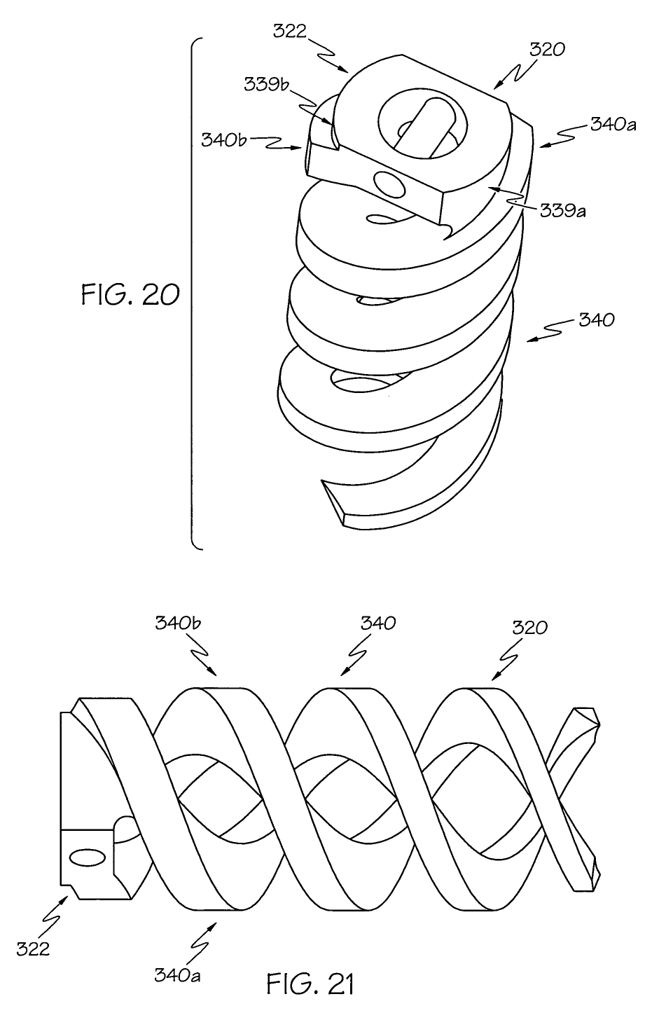

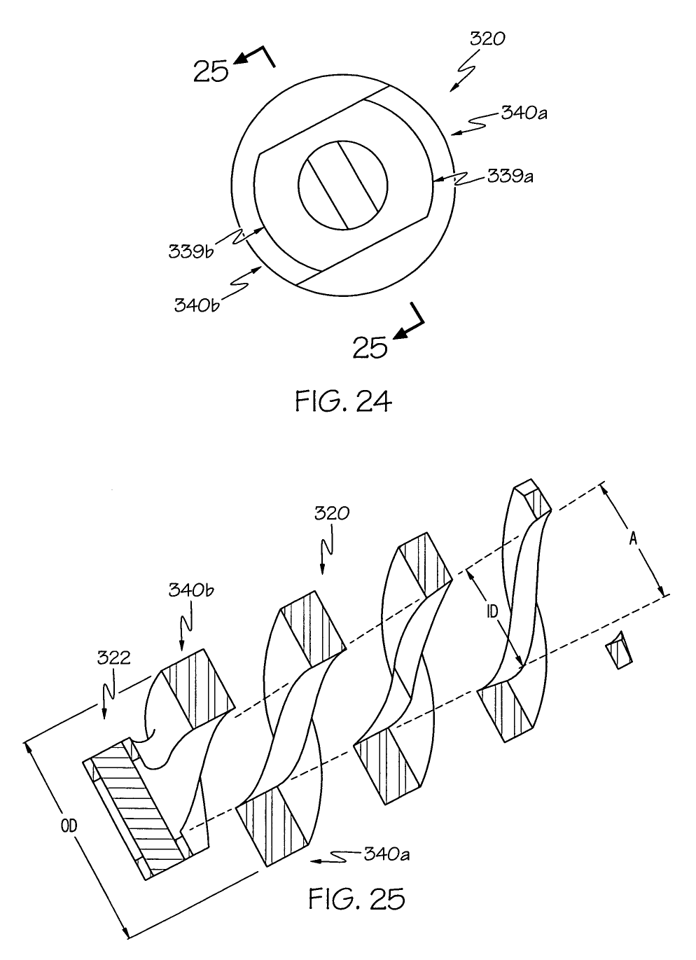

FIGS. 20-25 illustrate views of a helical anchor 320 in accordance with a fourth embodiment of the present invention. The helical anchor 320 can include similar and/or identical features described and illustrated with respect to the helical anchors 20, 120 and/or 220. For example, although not required, the head 322 of the helical anchor 320 can have similar or identical features as the head 122 of the helical anchor 120 described above.

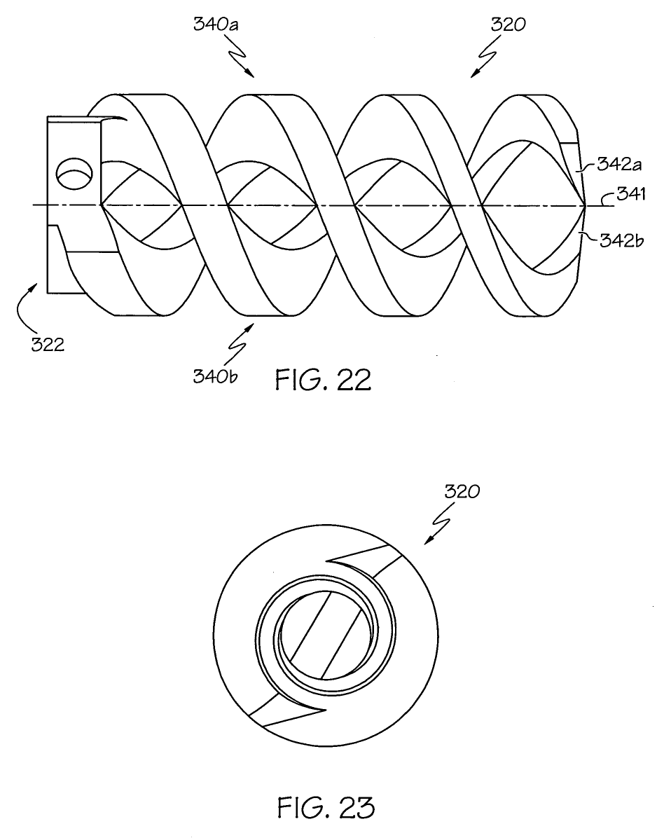

In accordance with aspects of the present invention, helical anchors can include one or more helical structures disposed about an axis and extending from the head. For example, as shown in FIG. 22, the helical anchor 320 includes two helical structures 340a, 340b disposed about an axis 341 and extending from the head 322. In the illustrated example, each helical structure 340a, 340b is substantially identical to the helical structure 140 of the helical anchor 120. Each helical structure begins at a first end portion attached to the head and a ends at a second end portion. Moreover, as shown in the figures, each end portion can be respectively attached at opposite peripheral locations 239a, 239b of the head 322 to provide a double helix configuration as shown in FIG. 21.

As best illustrated in FIG. 25, the helical structures 240a, 240b can each include a common, constant outer diameter “OD” and a changing inner diameter “ID” similar to the previously-described embodiments. The helical structures 240a, 240b further include a cross sectional area that reduces in cross section substantially continuously in a direction from the first end portion to the second end portion of the helical structures 340a, 340b. Providing the helical structures with a reduction in cross section can be achieved in a variety of ways as described and illustrated with respect to the helical structures 40, 140, 240.

While the illustrated examples of helical anchors depict a single or double helical structure, it is contemplated that three or more helical structures may be provided in accordance with aspects of the present invention. Moreover, each helical anchor may have different dimensions. For example, the overall length of the helical anchor along the axis can comprise about 10 to about 12 millimeters although other lengths are contemplated. Moreover, the “OD” of the helical structure can comprise about 5 millimeters although other diameters are contemplated. Moreover, although not limited to any particular dimensions, helical anchors can be constructed with dimensions set forth in U.S. Provisional Application No. 60/613,004 filed Sep. 24, 2004, which is incorporated by reference herein.

Anchors in accordance with the present invention may also be made from a wide range of materials adapted to provide sufficient structural integrity for anchor while providing a high implant material quality over time. In one particular example, the material can comprise implant quality 6AL-4V ELI titanium, or the like. The material can also comprise stainless steel, such as 316 stainless steel. Still further, other implant quality materials may be employed such as metals, plastics, bioresorbable materials (e.g., bioresorbable polymers), composites or other materials.

The helical anchors may also be formed by a variety of methods. In one example, the anchor is made from a bar stock of material that is machined using subtractive machining processes. In particular, machining processes including turning and milling techniques may be employed. If using a turning technique, costs associated with producing the anchor may be minimized by providing the helical structure with a constant outer diameter as illustrated throughout the embodiments herein. Moreover, the process may employ a subtractive process to form a rough anchor that is later refined using further machining techniques to fine-tune features of the anchor. For example, subtractive techniques may be used to create a rough anchor 220 as illustrated in FIGS. 14-19. Subsequent machining processes can fine-tune the features of the rough anchor 220 to obtain the anchor 20 illustrated in FIGS. 1-7. Similar machining techniques may also be used to produce the helical anchors 120, 320 illustrated in FIGS. 8-13 and FIGS. 20-25 described above.

A method of installing the anchor in accordance with the embodiments described above will now be described. Although the method is described with respect to the embodiment of FIGS. 1-7, it is understood that the method may be used with other helical anchors in accordance with aspects of the present invention. Moreover, although the method is described with respect to mounting to a bone tissue, it is understood that the method can equally apply to other tissue structure. Still further, although the method is described by attaching a suture to the anchor, it is understood that other elements can be attached to the anchor.

The suture may be attached to the anchor before or after the helical anchor is mounted to the bone tissue. For example, a tool may be designed to receive a suture, thereby simplifying the process of tying or otherwise securing the suture to the anchor. Thus, the suture may be initially looped around or tied to the crossbar 28 wherein the looped portions and/or tied portions adjacent the crossbar are protected by the shroud 24. Next, the distal, unattached end of the suture is threaded through an end of a tool. The head 22 of the anchor 20 is then inserted into the end of the tool such that two opposed prongs of the tool simultaneously engage the flat surfaces 36a, 36b of the head 22. Using the tool, the tip 43 of the anchor is then pressed against the bone tissue adjacent the mounting location. In certain procedures, a pilot hole may be drilled to provide an starting location for the tip 43 of the anchor 20. At least portions of the tool are rotated together with the helical anchor 20 to screw the helical anchor into the bone tissue. The helical anchor 20 may be screwed into the bone tissue until the head 22 is substantially adjacent with the exterior surface of the bone tissue. Next, the tool is removed wherein the suture is released from the interior portion of the tool. The suture may then be used to fasten tissue with respect to the bone tissue.

In another option, the bone can formed with a countersunk portion that is adapted to receive at least a portion of the head 22 of the anchor 20. The countersunk portion can be formed prior to mounting the anchor 20 to the bone tissue or can be formed as the anchor is being mounted to the bone tissue. The anchor 20 can be mounted such that the head 22 is located at least partially within the countersunk portion such that the top surface 34 of the head 22 is substantially flush or adjacent to the outer surface of the bone tissue. In particular, the top surface 34 may be flush with the outer surface of the bone tissue or can extend slightly above or below the bone surface. In these countersunk positions, the rounded inner periphery 30 of the shroud 24 can protect the suture from engaging adjacent sharp edges of the bone tissue.

From the above description of the invention, those skilled in the art will perceive improvements, changes and modifications. Such improvements, changes and modifications within the skill of the art are intended to be covered by the appended claims.

1. An anchor for mounting to a tissue structure comprising:
a head including a shroud encircling an interior area, the shroud including an outer periphery with two substantially flat surfaces that are substantially parallel with respect to one another, and a crossbar having first and second ends attached to the shroud such that the crossbar extends within the interior area between the two substantially flat surfaces; and
a fastening structure attached to the head and configured for mounting to a tissue structure.

2. The anchor of claim 1, wherein the fastening structure comprises a helical structure.

3. The anchor of claim 2, wherein the helical structure is disposed about an axis, the helical structure including a first end portion attached to the head and a second end portion including a distal end of the helical structure, the helical structure including a cross section that decreases from the first end portion to the second end portion, where the perimeters of each cross section are mathematically similar to one another.

4. The anchor of claim 2, wherein the helical structure is disposed about an axis, the helical structure including a first end portion at least partially attached to the outer periphery of the shroud and extending away from the outer periphery.

5. The anchor of claim 1, wherein the shroud includes a pair of arcuate wall structures facing the interior area wherein the interior area has a substantially oblong shape.

6. The anchor of claim 1, wherein a top surface of the head includes an opening in communication with the interior area, wherein an inner periphery of the head is rounded adjacent the opening of the top surface.

7. (canceled)

8. The anchor of claim 3, wherein the cross section substantially continuously decreases from the first end portion to the second end portion.

9. The anchor of claim 3, wherein each of the perimeters comprise a polygonal shape.

10. The anchor of claim 9, wherein each of the perimeters comprise a four-sided polygonal shape.

11. The anchor of claim 3, wherein the helical structure includes an axial thickness that decreases from the first end portion to the second end portion.

12. The anchor of claim 11, wherein the axial thickness substantially continuously decreases from the first end portion to the second end portion.

13. The anchor of claim 11, wherein the helical structure includes a radial thickness that decreases from the first end portion to the second end portion.

14. The anchor of claim 3, wherein the helical structure includes a radial thickness that decreases from the first end portion to the second end portion.

15. The anchor of claim 14, wherein the radial thickness substantially continuously decreases from the first end portion to the second end portion.

16. The anchor of claim 3, wherein the helical structure includes a length, and outer diameter and an inner diameter, wherein the outer diameter of the helical structure is substantially constant throughout substantially the entire length of the helical structure and the inner diameter of the helical structure increases substantially continuously throughout the entire length of the helical structure to define a frustoconical cavity.

17. A helical anchor comprising:
a head; and
a helical structure having a length and disposed about an axis, the helical structure including a first end portion attached to the head and a second end portion including a distal end of the helical structure, wherein the helical structure includes a cross section and an axial thickness that each decrease from the first end portion to the second end portion.

18. The helical anchor of claim 17, wherein the outer diameter of the helical structure is substantially constant throughout substantially the entire length of the helical structure and an inner diameter of the helical structure increases substantially continuously throughout the entire length of the helical structure to define a frustoconical cavity.

19. The helical anchor of claim 17, wherein parameters of each cross section are mathematically similar to one another.

20. The helical anchor of claim 17, wherein the cross section and the axial thickness each decrease substantially continuously throughout substantially the entire length of the helical structure.
